(19) United States
(12) Patent Application Publication
(10) Pub. No.: US 2008/0243252 A1
(43) Pub. Date: Oct. 2, 2008

(54) Spinal implant system

(75) Inventors: Eric Hansen, Arlington, TX (US); James Scott Hay, Parkland, FL (US)

Correspondence Address: CHRISTOPHER & WEISBERG, P.A., 200 EAST LAS OLAS BOULEVARD, SUITE 2040, FORT LAUDERDALE, FL, 33301, US

(73) Assignee: Centra-Fuse, Inc., Reno, NV (US)

(21) Appl. No.: 11/732,137

(22) Filed: Apr. 2, 2007

(51) Int. Cl. A61F 2/44

(52) U.S. Cl. 623/17.16; 623/17.11

(57) ABSTRACT

The present invention provides an intervertebral prosthesis generally defining a first wall, a second wall, sidewalls extending from the first wall to the second wall, and an upper surface and a lower surface. One or more passages may be included providing for an affixation element, to be inserted therethrough. The passages may each include a first portion for receiving a head or tip of an affixation element, and a second portion of a smaller diameter, where a substantial amount of the second portion of the passage located above or below a midline of the prosthesis. One or more openings may also be provided on the spinal prosthesis for coupling the prosthesis to a surgical tool, and the upper and lower surfaces of the prosthesis may include a depression or portion void of any teeth for the insertion of the prosthesis where a distraction tool is situated about the surgical site.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a method and system for replacing diseased and/or damaged intervertebral discs, and in particular, provides an artificial intervertebral disc prosthesis.

### BACKGROUND OF THE INVENTION

Disease, advancing age, and trauma can lead to changes in various bones, discs, joints, and ligaments of the body. Some changes and trauma often manifest themselves in the form of damage or degeneration to a spinal disc or portion thereof. Such conditions often lead to chronic back pain, which can range from mild discomfort to severe, debilitating conditions. Treatment for degeneration and/or disease may include spinal fusion of two adjacent vertebral bodies, which are joined together after removing the intervening intervertebral disc. Typically, a prosthetic device is positioned between the two adjacent vertebral bodies in place of the removed disc to subsequently fill the space left by the removed disc, which may allow bone to grow between the two vertebral bodies.

The success or failure of a spinal fusion procedure can often depend upon the particular characteristics of the prosthesis that is placed between the adjacent vertebral bodies. In addition to having sufficient structural integrity to withstand the repeated and varying loads experienced in the spine from day to day, the prosthesis must be biocompatible, and further, the prosthesis should permit the ingrowth of bone to complete the fusion. Furthermore, the prosthesis should have characteristics that permit the prosthesis to remain fixed in the desired position, and to further resist movement due to shifting, rotation, or slippage of the prosthesis.

Despite known prostheses for the fusion of adjacent vertebral bodies, there remains a need for additional prostheses that have desirable geometries and characteristics for achieving secure and effective implantation within a patient's spine.

### SUMMARY OF THE INVENTION

The present invention provides an intervertebral prosthesis having desirable geometries and characteristics for achieving secure and effective implantation within a patient's spine. In particular, a spinal prosthesis is provided generally defining a first wall, a second wall, and sidewalls extending from the first wall to the second wall. The spinal prosthesis may further define an upper surface and a lower surface, each of which may extend across a substantial portion of the body of the prosthesis. The spinal prosthesis may include somewhat of an “X” shaped cross section to provide improved resistance against compression forces experienced once implanted. In addition, an aperture may extend through a portion of the prosthesis from the upper surface to the lower surface, where the aperture may provide a path for bone growth and/or may allow for the addition of therapeutic materials within a portion of the prosthesis upon implantation. The surfaces and walls of the prosthesis may define a geometric profile of the spinal prosthesis.

The spinal prosthesis may further define one or more passages providing for an affixation element, such as a screw or nail, to be inserted therethrough. The passages may each include a first portion for receiving a head or tip of an affixation element, and a second portion of a smaller diameter or dimension than the first portion for accommodating a threaded or smaller portion of the affixation element. Moreover, each passage may be situated with a substantial amount of the second portion of the passage located above or below a midline of the prosthesis in order to minimize the amount of tissue from the surrounding area required for removal in order to guide a fixation element into the one or more passages at the desired angle upon implantation. The one or more passages of the prosthesis may further be angled to provide for increased pull strength to securely anchor the prosthesis and to further resist movement of an inserted affixation element when implanted.

One or more openings may also be provided on the spinal prosthesis for coupling the prosthesis to a surgical tool to aid in the placement and manipulation of the prosthesis during a particular procedure. In addition, the upper and lower surfaces and/or a portion of the first and second walls of the prosthesis may include a depression or surface void of any teeth for the insertion of the prosthesis where a distraction tool is situated about the surgical site.

The present invention may further include one or more sizing elements similarly shaped to the spinal prosthesis for determining the suitable dimensions of the spinal prosthesis to be used. In particular, the sizing elements may each include a sizing element body defining a first wall, a second wall, a pair of sidewalls, and upper and lower surfaces. The sizing element bodies may accordingly define a geometric profile substantially similar to the geometric profile of the spinal prosthesis, as described above. The sizing elements may have varying dimensions corresponding to the available dimensions of a particular implant, such that the sizing elements can temporarily be placed within the implant site to verify that a particular width, height, or the like, will provide the desired anatomical result upon implantation of a spinal prosthesis having similar dimensions.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a perspective view of an embodiment of a spinal prosthesis in accordance with the present invention;

FIG. 2 is a bottom view of an embodiment of a spinal prosthesis in accordance with the present invention;

FIG. 3 is a side view of an embodiment of a spinal prosthesis in accordance with the present invention;

FIG. 4 is a cross-sectional view of an embodiment of a spinal prosthesis along line A-A of FIG. 3 in accordance with the present invention;

FIG. 5 is a top view of an embodiment of a spinal prosthesis in accordance with the present invention;

FIG. 6 is a cross-sectional view of an embodiment of a spinal prosthesis along line B-B of FIG. 5 in accordance with the present invention;

FIG. 7 is a front view of an embodiment of a spinal prosthesis in accordance with the present invention;

FIG. 8 is a rear view of an embodiment of a spinal prosthesis in accordance with the present invention;

FIG. 9 is a perspective view of an embodiment of a sizing element in accordance with the present invention;

FIG. 10 is a top view of an embodiment of a sizing element in accordance with the present invention;

FIG. 11 is a side view of an embodiment of a sizing element in accordance with the present invention;

FIG. 12 is a bottom view of an embodiment of a sizing element in accordance with the present invention;

FIG. 13 is a cross-sectional view of an embodiment of a sizing element along line C-C of FIG. 12 in accordance with the present invention;

FIG. 14 is a rear view of an embodiment of a sizing element in accordance with the present invention; and

FIG. 15 is a front view of an embodiment of a sizing element in accordance with the present invention;

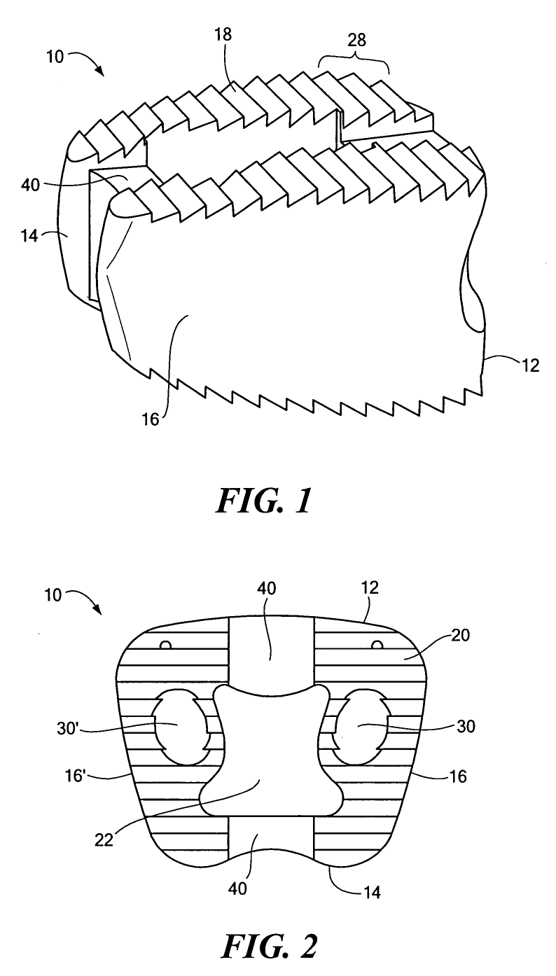

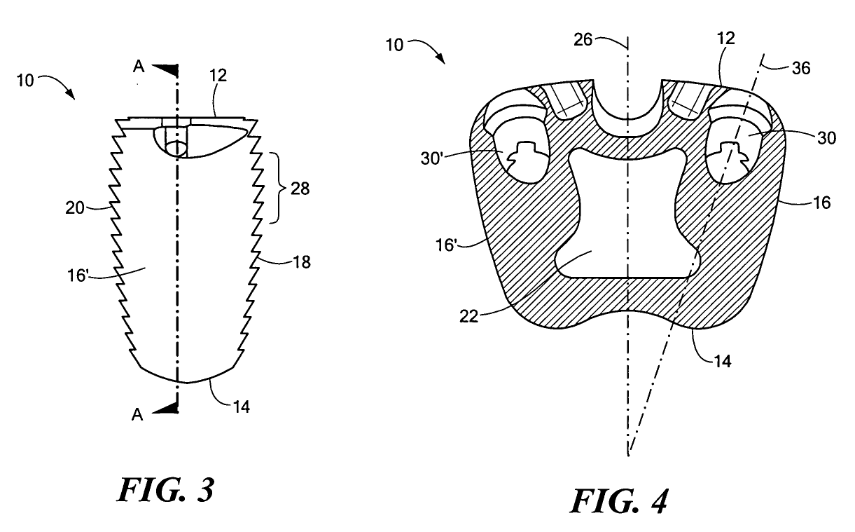

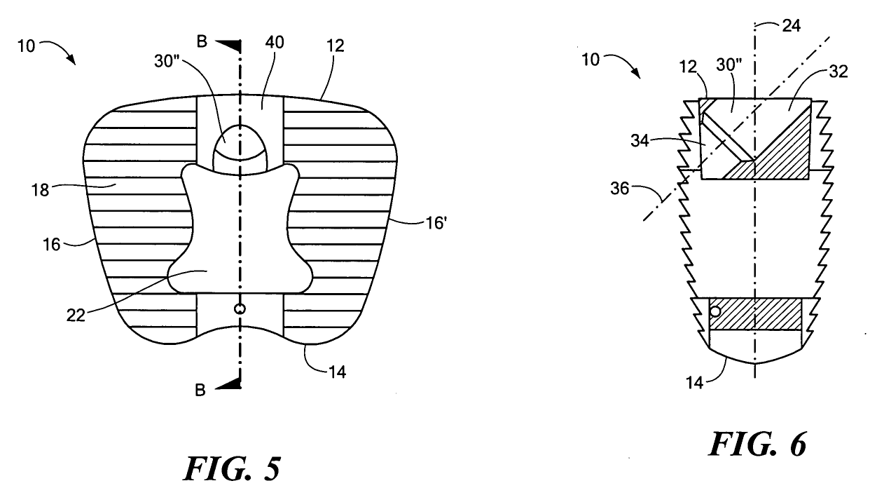

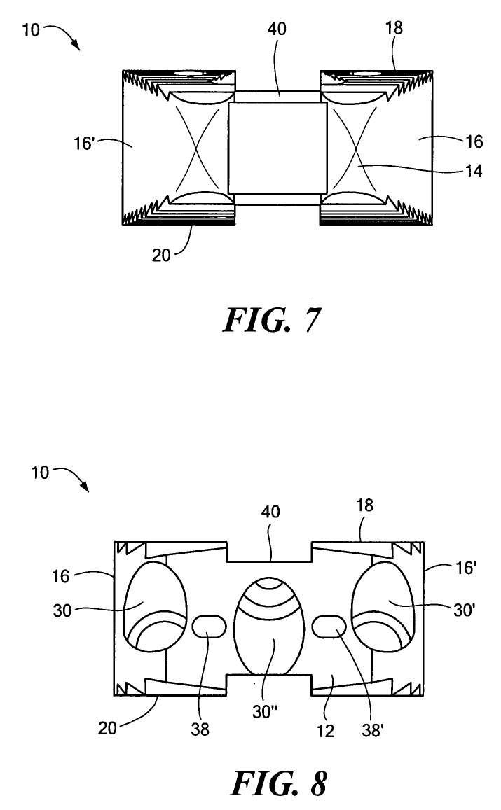

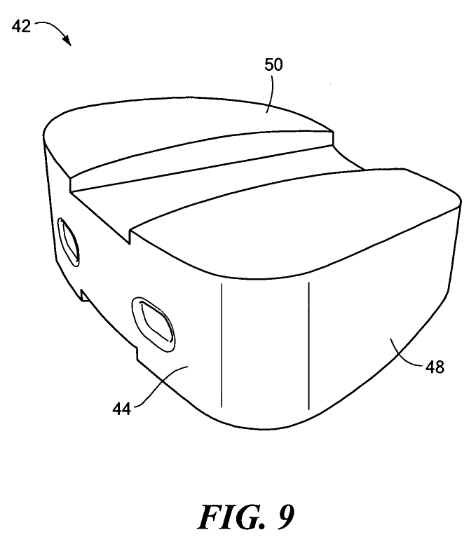

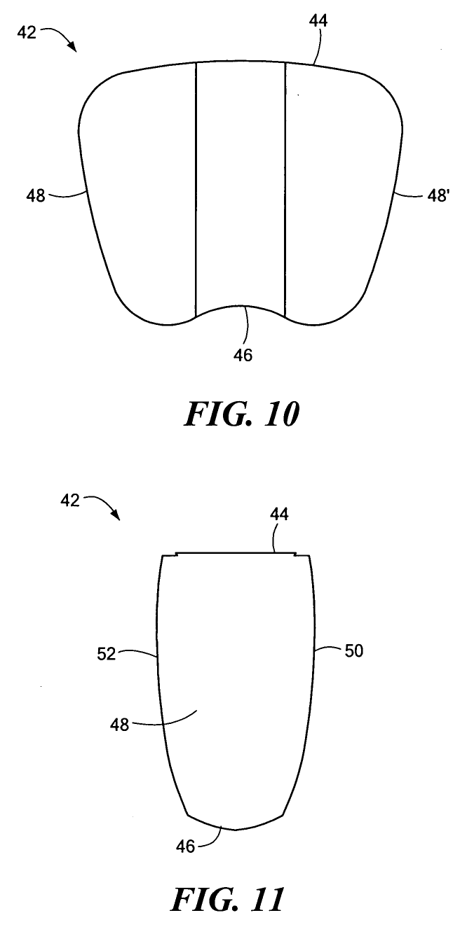

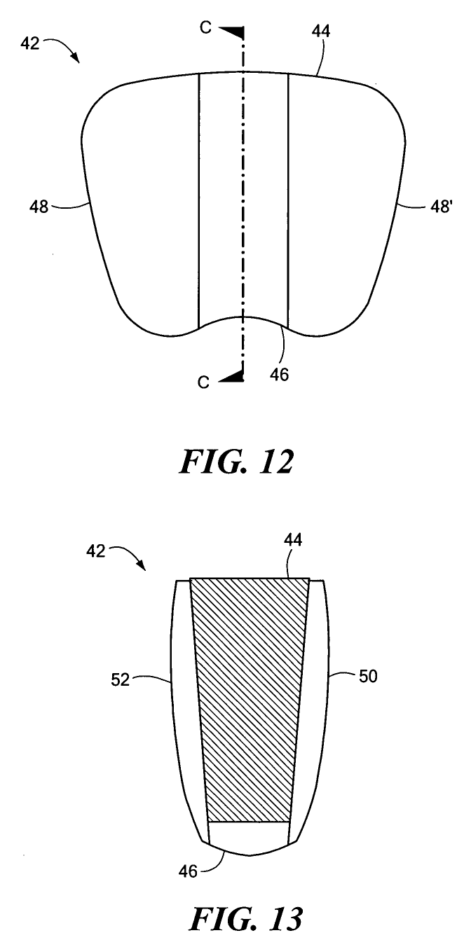

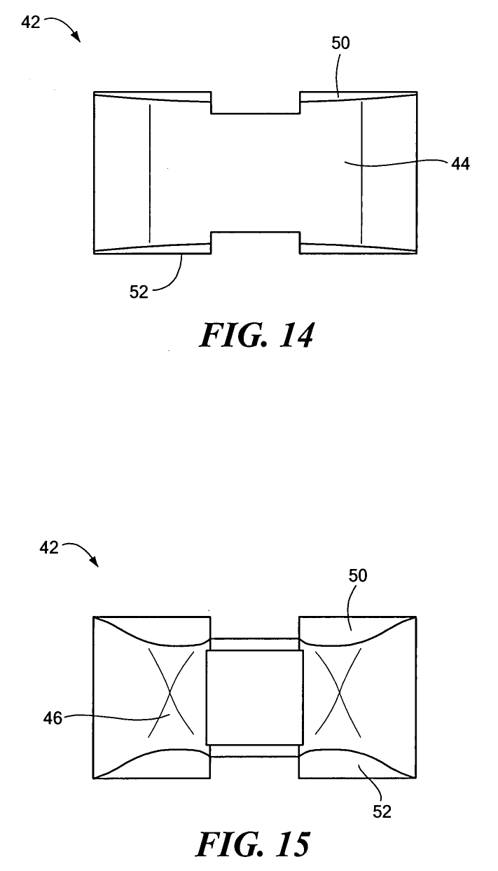

### DETAILED DESCRIPTION OF THE INVENTION

The present invention provides a system and method for repairing a diseased and/or damaged intervertebral disc or a portion thereof. In particular, a spinal prosthesis 10 is provided, where the spinal prosthesis 10 may include a prosthesis body generally defining a first wall 12, a second wall 14, and sidewalls 16,16′ extending from the first wall 12 to the second wall 14. The second wall 14 may be curved or rounded to reduce friction and thereby ease insertion of the spinal implant into a portion of the spinal column. The spinal prosthesis 10 may further define an upper surface 18 and a lower surface 20, each of which may extend across a substantial portion of the body of the prosthesis. In addition, an aperture 22 may extend through a portion of the prosthesis from the upper surface 18 to the lower surface 20, where the aperture 22 may provide a path for bone growth and/or may allow for the addition of therapeutic materials within a portion of the prosthesis upon implantation. The surfaces and walls of the prosthesis may define a geometric profile of the spinal prosthesis 10, where the geometric profile may include the particular dimensions of the surfaces involved, the angular orientation, and/or any curvature or arcuate orientation of a portion of the prosthesis. For example, the walls may define a particular curved or arcuate periphery, while the height of the walls may include a particular height desired for implantation.

The prosthesis may define a midline 24 positioned halfway in between the upper and lower surfaces of the prosthesis, as well as a centerline 26 positioned halfway between the sidewalls 16,16′ of the prosthesis. The second wall 14 of the prosthesis may define a height smaller than a height defined by the first wall 12, such that the upper and/or lower surfaces of the prosthesis are angled in a nonparallel configuration with respect to each other. Moreover, the upper and/or lower surfaces may have an arcuate shape extending between the first and second walls. In addition, the spinal prosthesis 10 may include a plurality of teeth 28 on a portion of the upper and lower surfaces to resist movement of the prosthesis once implanted into the desired position. The teeth may be sized or dimensioned to provide sufficient friction against movement of the implant upon implantation while reducing the likelihood that the teeth extend too far into the endplates of the surrounding vertebral bodies as to cause tissue damage.

The spinal prosthesis 10 may further define one or more passages providing for an affixation element, such as a screw or nail (not shown), to be inserted therethrough. Although not illustrated, the passages may be threaded or include a contoured or textured surface for securely receiving an affixation element. In particular, the spinal prosthesis 10 may include a first and second passage 30,30′ extending from a portion of the upper surface 18 proximate to the first wall 12, through the lower surface 20 and towards the second wall 14. In addition, the prosthesis may include a third passage 30″ extending from a portion of the lower surface 20 proximate to the first wall 12, and through the upper surface 18 towards the second wall 14. The passages may each include a first portion 32 for receiving a head or tip of an affixation element, and a second portion 34 of a smaller diameter or dimension than the first portion for accommodating a threaded or smaller portion of the affixation element. Moreover, each passage may be situated with a substantial amount of the second portion 34 of the passage located above or below a midline 24 of the prosthesis in order to minimize the amount of tissue from the surrounding area required for removal in order to guide a fixation element into the one or more passages at the desired angle upon implantation. In particular, the first and second passages 30,30′ may have a substantial part of their respective second portions located below the midline 24 as they extend downward towards the lower surface 20 in a direction towards the second wall 14, while the third passage 30″ may include a substantial part of the second portion located above the midline 24 towards the upper surface 18.

The one or more passages of the prosthesis may further be angled to provide for increased pull strength to securely anchor the prosthesis and to further resist movement of an inserted affixation element when implanted. For example, the first, second, and/or third passages may be oriented between the upper and lower surfaces of the prosthesis such that a longitudinal axis 36 extending through each of the respective passages is at an angle with the midline 24 of the prosthesis between approximately 35 degrees and approximately 45 degrees. Moreover, the longitudinal axis of one or more of the passages may define an angle with the centerline 26 of the prosthesis between approximately 10 degrees and 20 degrees as the passages extend from the first wall 12 to the second wall 14. As a result, the angular orientation of the one or more passages increases the cross-section of bone engaged to enhance the pull strength of an affixation element, as the angular orientation of three passages, for example, creates a triangular “boundary” of engaged bone to thereby secure the prosthesis in the desired location.

One or more openings may also be provided on the spinal prosthesis 10 for coupling the prosthesis to a surgical tool to aid in the placement and manipulation of the prosthesis during a particular procedure. For example, the first wall 12 of the prosthesis may include a first and second opening 38,38′ into a cavity and/or depression into the body of the prosthesis, which may include threading or other coupling elements to improve the grip and engagement between a tool and the implant. The openings and resulting cavity may further be oriented at an angle with the centerline 26 of the prosthesis, and may also be curved inward from the first and second openings 38,38′ towards the centerline 26 to further enable the coupling and/or grasping of the prosthesis by a surgical tool during insertion and positioning. In addition and/or alternatively, a positioning or gripping tool may engage or otherwise couple to the implant through the one or more passages that will ultimately receive an affixation element such as a pedicle screw or the like.

The upper and lower surface 20s and/or a portion of the first and second walls of the prosthesis may include a depression or surface 40 void of any teeth 28 for the insertion of the prosthesis where a distraction tool is situated about the surgical site. In a typical procedure, a spinal distraction tool is used to separate two intervertbral discs for the subsequent placement of a prosthesis. By providing a smooth and/or recessed portion on the prosthesis, the prosthesis may be maneuvered around a distraction tool already in place about the surgical site. As a result, the interference with the tool and surrounding tissue is minimized while the prosthesis 10 is maneuvered into a desired position. The smooth and/or recessed portion 40 of the prosthesis may be centered along the centerline 26 of the prosthesis, and may include dimensions adapted for receiving and/or being positionable about a spinal distraction tool.

The spinal prosthesis 10 may be constructed from a myriad of biocompatible materials providing desired mechanical behavior throughout the movement and resultant forces experienced in a spinal column. Moreover, the prosthesis 10 may include therapeutic substances disposed about the prosthesis, as well as imaging elements, such as radiopaque markers, embedded in portions of the prosthesis to aid in the positioning and/or monitoring of an implanted prosthesis.

The present invention may further include one or more sizing elements 42 similarly shaped to the spinal prosthesis 10 for determining the suitable dimensions of the spinal prosthesis 10 to be used. In particular, the sizing elements may each include a sizing element body defining a first wall 44, a second wall 46, a pair of sidewalls 48,48′, and upper and lower surfaces 50,52. The sizing element bodies may accordingly define a geometric profile substantially similar to the geometric profile of the spinal prosthesis 10, as described above. The sizing element 42 may have varying dimensions corresponding to the available dimensions of a particular implant, such that the sizing element 42 can temporarily be placed within the implant site to verify that a particular width, height, or the like, will provide the desired anatomical result upon implantation of a spinal prosthesis 10 having similar dimensions. Similar to that of the spinal prosthesis 10 discussed above, each sizing element 42 may include on or more opening or depressions in a surface thereof for coupling to a surgical tool and/or for positioning the sizing element about a previously positioned spinal distraction tool located about the surgical area. Further, the sizing element 42 may be substantially void of any teeth or other features for resisting movement within the implant region, as they are intended to only be positioned temporarily within a patient.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A spinal prosthesis, comprising:
a prosthesis body defining a first wall, a second wall, a pair of sidewalls, an upper surface, and a lower surface; wherein the prosthesis body defines a midline extending from the first wall and second wall and centered between the upper surface and lower surface;
a passage for receiving an affixation element, wherein the passage defines a first portion and a second portion, the second portion having a width less than the first portion, and wherein a substantial part of the second portion of the passage is located on one side of the midline.

2. The spinal prosthesis according to claim 1, wherein the passage defines a longitudinal axis, and wherein the longitudinal axis intersects the midline at an angle greater than approximately 35 degrees.

3. The spinal prosthesis according to claim 2, wherein the longitudinal axis intersects the midline at an angle less than approximately 45 degrees.

4. The spinal prosthesis according to claim 3, wherein the prosthesis body further defines a centerline centered between the sidewalls extending from the first wall to the second wall, wherein the longitudinal axis of the passage intersects the centerline at an angle greater than approximately 10 degrees.

5. The spinal prosthesis according to claim 4, wherein the longitudinal axis intersects the centerline at an angle less than approximately 20 degrees.

6. The spinal prosthesis according to claim 1, wherein the prosthesis body further defines a first opening on the first wall for receiving a tool therein.

7. The spinal prosthesis according to claim 6, further comprising a second opening on the first wall for receiving a tool therein; wherein the first and second openings are in fluid communication with first and second cavities, respectively, wherein the first and second cavities curve inward towards a centerline of the prosthesis.

8. The spinal prosthesis according to claim 1, further comprising a plurality of teeth on the upper and lower surfaces of the prosthesis body.

9. The spinal prosthesis according to claim 8, further comprising a portion on one of the upper and lower surfaces void of teeth.

10. The spinal prosthesis according to claim 1, further comprising a plurality of radiopaque markers disposed about the prosthesis body.

11. A spinal implant system, comprising:
a spinal prosthesis defining a first geometric profile;
a sizing element defining a second geometric profile, wherein the second geometric profile is substantially similar to the first geometric profile.

12. The spinal implant system according to claim 1 1, wherein the spinal prosthesis defines a first wall, a second wall, and two sidewalls, and wherein the first geometric profile includes a peripheral shape defined by the first wall, second wall, and two sidewalls.

13. The spinal implant system according to claim 11, wherein the spinal prosthesis defines an upper surface, a lower surface, and a prosthesis height defined therebetween, and wherein the first geometric profile includes the prosthesis height.

14. The spinal implant system according to claim 13, further comprising a plurality of teeth on the upper and lower surfaces of the prosthesis body.

15. The spinal implant system according to claim 14, further comprising a portion on one of the upper and lower surfaces of the prosthesis being substantially void of teeth.

16. The spinal implant system according to claim 15, wherein the sizing element includes an upper surface, a lower surface, and wherein the upper and lower surfaces each define a recessed portion.

17. The spinal implant system according to claim 11, further comprising a plurality of radiopaque markers disposed about the spinal prosthesis.

18. A spinal implant system, comprising:
a spinal prosthesis defining a first wall, a second wall, a pair of sidewalls, an upper surface, and a lower surface, wherein the walls and upper and lower surfaces define a first geometric profile; wherein the prosthesis body defines a midline extending from the first wall and second wall and centered between the upper surface and lower surface; the spinal prosthesis further defining a passage for receiving an affixation element, wherein the passage defines a first portion and a second portion, the second portion having a width less than the first portion, and wherein a substantial part of the second portion of the passage is located on one side of the midline; and
a sizing element defining a second geometric profile, wherein the second geometric profile is substantially similar to the first geometric profile.

19. The spinal implant system according to claim 18, further comprising a plurality of teeth on the upper and lower surfaces of the spinal prosthesis.

20. The spinal implant system according to claim 19, further comprising a portion on one of the upper and lower surfaces of the spinal prosthesis being substantially void of teeth.
